(19) United States
(12) Patent Application Publication
(10) Pub. No.: US 2003/0175206 A1
(43) Pub. Date: Sep. 18, 2003

(54) Liposomal encapsulation of chelated actinium-225 and uses thereof

(76) Inventors: George Sgouros, New York, NY (US); James L. Thomas, New York, NY (US); David A. Scheinberg, New York, NY (US); Michael R. McDevitt, Bronx, NY (US); Stavroula Sofou, New York, NY (US)

Correspondence Address: Benjamin Aaron Adler, ADLER & ASSOCIATES, 8011 Candle Lane, Houston, TX, 77071, US

(21) Appl. No.: 10/319,978

(22) Filed: Dec. 16, 2002

Related U.S. Application Data
(63) Continuation-in-part of application No. PCT/US01/19133, filed on Jun. 15, 2001.
(60) Provisional application No. 60/212,186, filed on Jun. 16, 2000.

(51) Int. Cl. A61K 51/00; A61K 9/127

(52) U.S. Cl. 424/1.11; 424/450

(57) ABSTRACT

The present invention provides targeted delivery of alpha particle-emitting radionuclides and their alpha-emitting progeny using liposomal encapsulation to prevent the loss of daughter radionuclides from the targeting vehicle and, therefore, from the tumor site.

This U.S. national application claims benefit of international application PCT/US01/19133, filed Jun. 15, 2001, which claims benefit of provisional patent application U.S. Ser. No. 60/212,186, filed Jun. 16, 2000, now abandoned.

### BACKGROUND OF THE INVENTION

1. Field of the Invention

The present invention relates generally to the field of radiotherapy. More specifically, the present invention relates to liposomal encapsulation of alpha particle-emitting radionuclides. Most specifically, the present invention relates to liposomal encapsulation of chelated actinium-225 and uses thereof.

2. Description of the Related Art

Optimal treatment with many drugs requires maintenance of a drug level for an extended period of time. For example, optimal anti-cancer treatment with cell cycle-specific antimetabolites requires maintenance of a cytotoxic drug level for a prolonged period of time. The half-life of many drugs after an intravenous (IV), subcutaneous (SC), intraperitoneal (IP), intraarterial (IA), intramuscular (IM), intrathecal (IT), or epidural dose is very short, being in the range of a fraction of an hour to a few hours.

Epithelial ovarian cancer is described as the “silent killer” because in the majority (70%) of patients the disease is first detected as a result of symptoms arising after the disease has spread outside of the pelvis and into the peritoneal cavity. In such cases of advanced disease (FIGO-Stage III), the 5-year survival rate employing current treatment approaches is approximately 15 to 20%. Disseminated ovarian carcinoma is rarely cured by current treatment options. Thus a new treatment modality is needed for disseminated epithelial ovarian carcinoma or other cancers.

Alpha particle-emitting radionuclides are effective cytotoxic agents, capable of tumor-cell kill without limiting morbidity and, thus, hold great promise as potential therapeutic agents for disseminated disease such as in cancer treatment. Due to their high energy deposition per distance traveled, alphas are capable of sterilizing individual cells or cell clusters with only one to three traversals through the nucleus. In addition, the 50-100 μm range of alphas is consistent with the dimensions of disseminated disease, allowing for localized irradiation of target cells with minimal normal cell irradiation.

The radionuclides, astatine-211 (At-211) and bismuth-213 (Bi-213) have been investigated clinically⁴⁻⁶. Both have short half-lives, 7 hours and 46 minutes, respectively, and are, therefore, appropriate for situation in which targeting is very rapid. The first injection of an alpha-particle emitter to humans for radioimmunotherapy (RIT) was with ²¹³Bi conjugated to the anti-CD33 antibody, HuM195, targeting myeloid leukemia⁴. This trial demonstrated feasibility and anti-cancer activity with minimal toxicity. In the second of these human trials the anti-tenascin antibody, 81C6, labeled with the alpha-particle emitter ²¹¹At, was injected into surgically created cavities in the patients with malignant gliomas. This trial has demonstrated substantially better tumor control relative to ¹³¹I-labeled 81C6 antibody ⁶. Animal studies have shown that alpha-particle emitters yield superior tumor control relative to beta or Auger electron emitters ⁷⁻¹¹.

In treating dissemated diseases or cancer, e.g., late-stage breast cancer with measurable liver or bone metastases, longer-lived alpha particle emitters are required to reach distant metastases that have developed their own vasculature. The effectiveness of alpha particles arises because the amount of energy deposited per unit distance traveled, the linear energy transfer or LET, is approximately 400 times greater than that of beta particles, i.e., 80 keV/μm vs. 0.2 keV/μm. Cell survival studies have shown that alpha-particle induced killing is independent of oxygenation state or cell-cycle during irradiation and that as few as 1 to 3 tracks across the nucleus may result in cell death¹⁻³.

One of the most promising alpha-particle emitting radionuclides, Ac-225 has a 10-day half-life and, therefore, unlike ²¹³Bi (45.6-min half-life) and ²¹¹At (7.2 h half-life) is not limited to the targeting of rapidly accessible disease. Each decay of ²²⁵Ac leads to the emission of four alpha particles (FIG. 1). Studies, in vitro, have shown that this radionuclide is approximately 1000-fold more effective than the first generation alpha-emitter, ²¹³Bi that is currently under clinical investigation. Studies in animals, however, have also shown that it is substantially more toxic. The increased efficacy and toxicity are a result of the alpha-particle emitting intermediates. When these are confined to the target cells, efficacy is increased, when they distribute throughout the body, toxicity is increased.

This toxic potential presents a fundamental difficulty if antibody or other molecular approaches to delivery of this radionuclide are used since the bond between the targeting vehicle and the radionuclide is broken upon transformation of the parent and emission of the first alpha. This leaves the first daughter in the decay chain free to distribute throughout the body where it will decay and subsequently yield additional alpha emissions to normal organs from subsequent daughter decays. Of the 4 alphas, only the first one originating from decay of Ac-225 contributes to the tumor dose, the remainder will distribute throughout normal tissue to increase toxicity. The radiotoxicity of the Ac-225 daughter isotopes is a limiting factor in radioimmunological therapies. This problem is lessened if the daughter radioisotope can be retained within a delivery vehicle. ¹²

Liposomes and their potential as drug-delivery vehicles have been investigated for many years. Liposomes are structures defined by a phospholipid bilayer membrane that encloses a n aqueous compartment. The membrane acts as a barrier that inhibits free molecular diffusion across the bilayer. Multivesicular liposomes (MVL), first reported by Kim, et al. (Biochim, Biophys. Acta, 728:339-348, 1983), are uniquely different from other lipid-based drug delivery systems such as unilamellar (Huang, Biochemistry, 8:334-352, 1969; Kim, et al., Biochim. Biophys. Acta, 646:1-10, 1981) and multilamellar (Bangham, et al., J Mol. Bio., 13:238-252, 1965) liposomes. In contrast to unilamellar liposomes (also known as unilamellar vesicles, or “ULV”), multilamellar and multivesicular liposomes (MVL) contain multiple aqueous chambers per particle. Because of the similarity in acronyms, multivesicular liposomes (MVL) are frequently confused with multilamellar liposomes (MLV). Nevertheless, the two entities are entirely distinct from each other. Whereas multilamellar liposomes (also known as multilamellar vesicles or MLV) contain multiple concentric chambers within each liposome particle, resembling the “layers of an onion,” the multiple aqueous chambers in multivesicular liposomes are non-concentric. The structural differences between unilamellar, multilamellar, and multivesicular liposomes are well known in the art.

The structural and functional characteristics of multivesicular liposomes are not directly predictable from current knowledge of ULV and multilamellar liposomes. The differences are described in the book Liposomes as Tools in Basic Research and Industry (Jean R. Philippot and Francis Schuber, eds., CRCpress, Boca Raton, Fla., 1995, pg. 19). Multivesicular liposomes are bounded by an external bilayer membrane shell, but have a very distinctive internal morphology, which may arise as a result of the special method employed in the manufacture. Topologically, multivesicular liposomes (MVL) are defined as liposomes containing multiple non-concentric chambers within each liposome particle, resembling a “foam-like” matrix. The presence of internal membranes distributed as a network throughout multivesicular liposomes may serve to confer increased mechanical strength to the vesicle, while still maintaining a high volume:lipid ratio as compared to multilamellar liposomes.

The multivesicular nature of multivesicular liposomes also indicates that, unlike in unilamellar vesicles, a single breach in the external membrane of a multivesicular liposome will not result in total release of the internal aqueous contents. Thus, both structurally and functionally the multivesicular liposomes are unusual, novel and distinct from all other types of liposomes. As a result, the functional properties of multivesicular liposomes are not predictable based on the prior art related to conventional liposomes such as unilamellar vesicles and multivesicular liposomes.

The prior art describes a number of techniques for producing unilamellar vesicles and multivesicular liposomes (for example, U.S. Pat. Nos. 4,522,803 to Lenk; 4,310,506 to Baldeschwieler; 4,235,871 to Papahadjopoulos; 4,224,179 to Schneider; 4,078,052 to Papahadjopoulos; 4,394,372 to Taylor; 4,308,166 to Marchetti; 4,485,054 to Mezei; and 4,508,703 to Redziniak). The prior art also describes methods for producing multivesicular liposomes (Kim, et al., Biochim. Biophys. Acta, 728:339-348, 1983). For a comprehensive review of various methods of unilamellar vesicles and multivesicular liposomes preparation, refer to Szoka, et al., Ann. Rev. Biophys. Bioeng.,9:465-508, 1980. In the method of Kim, et al. (Biochim. Biophys. Acta, 728:339-348, 1983), the pharmaceutical utility of multivesicular liposomes encapsulating small therapeutic molecules, such as cytosine arabinoside or cytarabine, is limited. Subsequent studies (Kim, et al., Cancer Treat. Rep., 71:705-711, 1987) showed that the release rate of encapsulated molecules into biological fluids can be modulated by encapsulating in the presence of a hydrochloride.

Although liposomes have been used in the delivery of chemotherapy and in gene targeting, the use of liposomes in the delivery of radioactivity has not been accepted. This is primarily because high uptake of the liposomes was observed in reticuloendothelial organs such as the liver in the spleen during initial studies performed in the 1980's. Since that time, however, new liposomal systems have been generated with reduced reticuloendothelial uptake. Examples include sterically-stabilized liposomes coated with monosialogangliosides or polyethylene glycol (PEG). The use of such liposomes for the delivery of actinium-225 (Ac-225) and other promising alpha emitters, such as radium-223 (Ra-223) is particularly compelling because they may retain daughter radionuclides with the aqueous phase and thereby reduce systemic toxicity. Since range of an alpha particle (50-100 microns) is sufficient to penetrate beyond the liposomal membranes (70 nm), tumor irradiation will be enhanced.

The inventors have recognized a need in the art for a n method of targeted delivery of alpha particle emitting radionuclides with improved retention of the daughter radionuclides within the delivery vehicle. Liposomal encapsulation presents a possible strategy for the delivery of actinium and it's daughters. The prior art is deficient in an effective means for sequestering Ac-225 and its daughter radionuclides at specific targets during radiotherapy. The present invention fulfills this long-standing need and desire in the art.

### SUMMARY OF THE INVENTION

In one embodiment of the present invention there is provided a method of preventing the systemic release of radioactive decay intermediates upon administration of an alpha particle-emitting radionuclide to an individual comprising the steps of incorporating the radionuclide into large liposomes having a diameter sufficient to retain the radioactive decay intermediates and administering the large liposomes to the individual such that the radioactive decay intermediates remain sequestered within the large liposomes.

In another embodiment of the present invention there is provided a method of targeting cells in an individual for liposomal delivery of an alpha particle-emitting radionuclide thereto without systemic release of radioactive decay intermediates comprising the steps of encapsulating the radionuclide within a small liposomal vesicle; incorporating the radionuclide into the aqueous phase of large liposomes having a diameter sufficient to encompass a cumulative recoil distance of all radioactive decay intermediates of said radionuclide. The liposome comprises polyethyleneglycol-linked lipids (PEG-lipids) on the outer membranes thereof and targeting agents specific to the cells attached to the PEG-lipids. The radionuclide is delivered to the cell whereby the targeting agents target the cells while the radioactive decay intermediates remain sequestered within the large liposomes.

Other and further aspects, features, and advantages of the present invention will be apparent from the following description of the embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate embodiments of the invention and therefore are not to be considered limiting in their scope.

FIG. 1 depicts the actinium-225 decay cascade with associated particulate decays and half lives.

FIG. 2 shows the radioactivity collected in each fraction after Sephadex™ column chromatography at different times after ¹¹¹In encapsulation.

FIG. 3 shows the retention of ¹¹¹In in liposomes as a function of time after loading.

FIG. 4 shows the model of Ac-225 radioactive decay used to determine loss rate of daughter radionuclides from liposome-encapsulated Ac-225 activity.

FIGS. 5A and 5B show sample simulations using models of the transfer rate from each sub-compartment within liposomes to the extraliposomal compartment.

FIGS. 6A-6D depict simulations obtained using four different loss rates.

FIGS. 7A and 7B show the relationship between loss rate and the levels of daughter activity at different measurement times after liposome separation.

FIG. 8 depicts theroetical model predictions of bismuth-213 retention for different liposome sizes. Vesicles prepared by extrusion through filters with larger pores show improved bismuth retention. Binding of radionuclides to the liposomal membrane (surface) significantly reduces retention.

FIG. 9A depicts the stability of zwitterionic liposomes. Fraction of fluorescence increase due to calcein leakage from PEGylated zwitterionic liposomes over time for liposomal diameters 800 nm (●), 400 nm (▾) and 100 nm (◯). After the first 24 hours a 20% fluorescence increase was measured for all liposomes, possibly due to differences in osmotic pressure across the liposomal membrane. Beyond this point all liposomes were stable for over 20 days. The error bars correspond to standard errors of repeated measurements.

FIG. 9B depicts the stability of positively charged liposomes. Fraction of fluorescence increase due to calcein leakage from PEGylated positive liposomes over time for liposomal diameters 800 nm (●), 400 nm (◯) and 100 nm (▾). The stability profiles resemble that of zwitterionic liposomes in FIG. 9A. The error bars correspond to standard errors of repeated measurements.

FIG. 10A depicts bismuth-213 activity in the pooled vesicle fractions of rechromatographed vesicles at several time points after preparation of the loaded vesicles. The bismuth-213 recovery curve can be well fit since the kinetics of the rise in bismuth activity are nearly monoexponential with a t½ equal to the bismuth half-ife. These vesicles were prepared by extrusion through 100 nm pore filters. The recovery of bismuth-213 activity to steady state levels is discussed the main text; the decrease in initial bismuth-213 activity reflects the rapid loss of bismuth from vesicles. Note that the vesicles entrap steady -state concentrations of all species in the decay chain.

FIG. 10B depicts leaking of bismuth-213 from liposomes. If bismuth-213 (dashed line) is not leaking (assume liposome separation at arrow position -a-) then the bismuth-213 activity concentration measured by gamma counting would be the same over several hours of measurement since it would be a t equilibrium with actinium-225 which has a 10 day half-life. Otherwise, the recovery curve of bismuth activity will follow almost monoexponential kinetis with t½ equal to the bismuth half-life assuming separation at the arrow position -b-.

FIG. 11A depicts actinium-225 retention for 100 nm (●), 400 nm (◯) and 800 (▾) nm zwitterionic liposomes produced by extended hydration over a period of 30 days. The error bars correspond to standard errors of repeated measurements.

FIG. 11B depicts actinium-225 retention for 100 nm (●), 400 nm (◯) and 800 (▾) nm positively charged liposomes produced by extended hydration over a period of 30 days. The error bars correspond to standard errors of repeated measurements.

FIG. 12A depicts bismuth-213 retention for 100 (●), 400 nm (◯) and 800 (▾) nm zwitterionic liposomes produced by extended hydration over a period of 30 days. The error bars correspond to standard errors of repeated measurements.

FIG. 12B depicts bismuth-213 retention for 100 (●), 400 nm (◯) and 800 (▾) nm positively charged liposomes produced by extended hydration over a period of 30 days. The error bars correspond to standard errors of repeated measurements.

FIG. 13 depicts multivesicular liposomes which consist of smaller liposomes loaded with actinium-225 entrapped into the larger structures.

FIG. 14 depicts the parallel elution profiles of large (triangles) and small (circles) liposomes in S-1000 column.

FIG. 15A depicts calcein quenching ratios for each eluted fraction from S-1000 in MUVELs. Calcein was entrapped at self-quenching concentrations only in the small liposomes (open symbols as in FIG. 14).

FIG. 15B depicts total entrapped calcein concentration (after addition of Triton x-100) on each MUVEL fraction from S-1000 (closed symbols; open symbols as in FIG. 14).

FIG. 16 depicts calcein quenching ratios of fractions 15, 16, 17, 18 from S-1000 over four days. Day 1: open symbols. Day 4: closed symbols.

FIG. 17 depicts the parallel elution profiles of large (800 nm filter diameter- closed symbols) and sonicated (open symbols) liposomes in S-1000 column.

FIG. 18A depicts the percentage of actinium retention in MUVEL fractions 8 (closed circles), 8.5 (open circles), 9 (closed triangles), and 15 (open triangles).

FIG. 18B depicts the percentage of bismuth retention in MUVEL fractions 8 (closed circles), 8.5 (open circles), 9 (closed triangles), and 15 (open triangles).

FIG. 19 is optical images of the peritoneum shown ventral view, head up eighteen hours after intraperitoneal injection of FITC-Herceptin. The perisplenic tumor (T), microscopic tumor modules (arrows) and urine in the bladder (B) which autofluoresces are seen on the fluorescent image in panel 2. Corresponding regions are identified on the bright field image in panel 1. This approach will be useful in evaluating the relative localization of Herceptin Ab vs. the Herceptin-coated immunoliposomes.

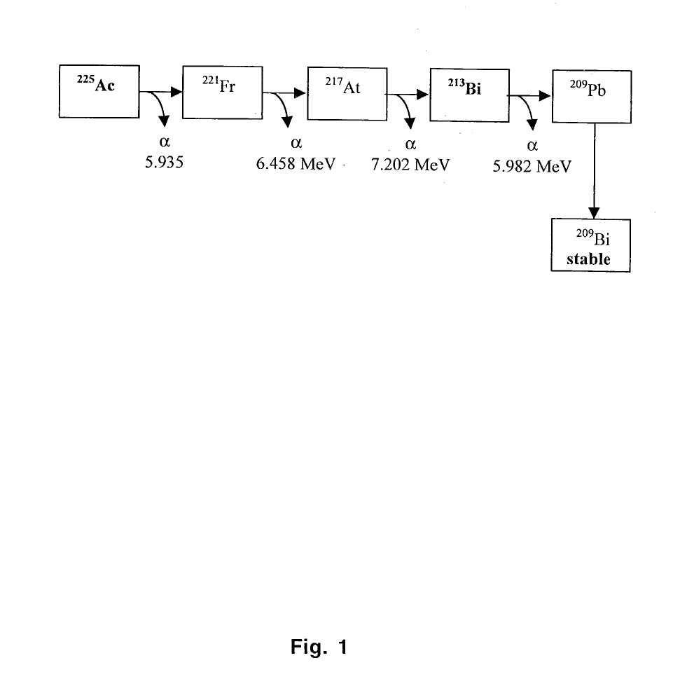

### DETAILED DESCRIPTION OF THE INVENTION

In one embodiment of the present invention there is provided a method of preventing the systemic release of radioactive decay intermediates upon administration of an alpha particle-emitting radionuclide to an individual comprising the steps of incorporating the radionuclide into large liposomes having a diameter sufficient to retain the radioactive decay intermediates and administering the large liposomes to the individual such that the radioactive decay intermediates remain sequestered within the large liposomes.

In one aspect of this embodiment the method further comprises the step of entrapping the radionuclide within a smaller liposomal vesicle prior to incorporating the radionuclide into the aqueous phase of the larger liposome. In another aspect of this embodiment the method further comprises preinjecting the individual with empty large liposomes and saturating the reticuloendothelial organs to reduce non-tumor specific spleen and liver uptake of the radionuclide upon adminstration thereof.

In yet another aspect of this embodiment the method further comprises coating the outer membrane surfaces of the large liposomes with molecules that preferentially associate with a specific target cell. These molecules or targeting agents may be antibodies, peptides, engineered molecules or fragments thereof. A representative example of such an antibody is Herceptin. The target cells may be cancer cells. Examples of such cancer cells are those found in ovarian cancer, breast cancer or metastatic cells thereof.

In all aspects of this embodiment the large liposomes have a diameter of about 600 nm to about 1000 nm. The large liposomes may further comprise molecules incorporated into the outer membranes to stabilize them. An example of such a molecules are polyethyleneglycol-linked lipids (PEG-lipids). These molecules may be used to attach to a targeting molecule as disclosed supra. The large liposomes disclosed herein may further comprise stabilizing agents or have an aqueous phase with a high pH. Representative examples of stabilizing agents are a phosphate buffer, an insoluble metal binding polymer, resin beads, metal-binding molecules or halogen binding molecules incorporated into the aqueous phase to further facilitate retention of the radioactive decay intermediates. Additionally, the large liposomes may comprise molecules to facilitate membrane fusion with the target cells or to facilitate endocytosis by the target cells.

Again in all aspects of this embodiment the alpha particle emitting radionuclide may be incorporated into the aqueous phase as a chelation compound with or without other stabilizing agents. Representative examples of this nuclide are ²²⁵Ac, ²²³Ra, ²¹³Bi ²¹²Pb, or ²¹¹At. A preferred radionclide is ²²⁵Ac.

In another embodiment of the present invention there is provided a method of targeting cells in an individual for liposomal delivery of an alpha particle-emitting radionuclide thereto without systemic release of radioactive decay intermediates comprising the steps of encapsulating the radionuclide within a small liposomal vesicle; incorporating the radionuclide into the aqueous phase of large liposomes having a diameter sufficient to encompass a cumulative recoil distance of all radioactive decay intermediates of said radionuclide. The liposome comprises polyethyleneglycol-linked lipids (PEG-lipids) on the outer membranes thereof and targeting agents specific to the cells attached to the PEG-lipids. The radionuclide is delivered to the cell whereby the targeting agents target the cells while the radioactive decay intermediates remain sequestered within the large liposomes. In all aspects of this embodiment further method steps, the radionuclides, the antibodies, targeting agents, the liposomes and the components thereof are as described supra.

The present invention provides targeted delivery of alpha particle-emitting radionuclides and their alpha-emitting progeny as it relates, for example, to cancer therapy using liposome-encapsulated alpha emitters. The systemic release of radioactive decay intermediates upon administration of an alpha particle-emitting radionuclide to an individual is prevented by incorporating the radionuclide into the liposome either in the aqueous phase or in association with the membrane. The liposomes are coated with molecules that preferentially associate with a target cell such as anti-tumor antibodies, peptides, engineered molecules or fragments thereof. Encapsulation of Ac-225 within an immunoliposome reduces loss of radioactive decay intermediates from the targeting vehicle and, by extension, the tumor site. The radioactive decay intermediates that remain sequestered within the multivesicular liposomes or MUVELs are not systemically released into the individual.

The alpha particle emitting radionuclide may be incorporated into the aqueous phase as a chelation compound. It is further contemplated to facilitate retention of radioactive decay intermediates by incorporating stabilizing agents, such as a phosphate buffer, insoluble metal binding polymer, resin beads, metal-binding molecules or halogen binding molecules, into the aqueous phase of the liposomes. Also, the pH of the liposome may be increased to facilitate retention. Additionally, it is contemplated that the instant liposomes further comprise additional molecules to facilitate membrane fusion with target cells or to facilitate endocytosis by target cells. The instant invention is especially useful for the delivery of the alpha particle-emitting radionuclides ²²⁵Ac, ²²³Ra, ²¹³Bi, ²¹²Pb, and ²¹¹At.

To increase confinement of these intermediate daughters to the targeted tumor site, engineered liposomes of sufficient diameter containing actinium-225 are used. Ac-225 retention is sufficient within the aqueous phase of the liposome. However, the radionuclides localize to the membrane surface of the liposomes, thus causing considerable decrease of the daughter retention. Because of the recoil distance of the daughter nuclei following alpha particle emission, 600 to 1000 nm-diameter liposomes are required in order to achieve adequate (>60%) daughter retention while still allowing tumor cell irradiation by alpha particles.

However, even with a large unilamellar liposome (LUV) having sufficient diameter membrane localization of the radionuclides is still a key parameter in these liposome systems with concomitant loss of daughters. To retain greater than 60% of bismuth-213 activity within the liposome, the actinium must be entrapped within the aqueous phase and not associated with the membrane surface of the LUV. Encapsulating the actinium-225 within a small liposome which is then entrapped within the aqueous phase of the larger liposomal structure forms multivesicular liposomes or MUVELs.

The instant invention is especially directed to the use of the liposomes for the delivery of alpha particle emitting radionuclides for the treatment of cancer. The relatively large size of liposomes, 600-1000 nm in diameter, that is required for adequate bismuth retention is advantageous for therapy of intraperitoneally disseminated ovarian cancer ²³. However, use of the liposomes in this context is not restricted to ovarian cancer and may be efficacious in treatment of, inter alia, breast cancer with measurable liver or bone metastases. The instant, invention also provides a method to reduce non-tumor specific uptake of the radionuclide containing liposomes into reticuloendothelial organs such as the spleen and liver by preinjecting empty liposomes into the individual to saturate absorption of liposomes into these organs.

To retain the intermediates at the tumor site, large (600-1000 nm in diameter) engineered liposomes with encapsulated ²²⁵Ac and immunolabeled with anti-tumor antibodies, e.g., Herceptin antibody, are provided. Aside from the critical advantage of daughter retention, liposomal delivery of alpha-emitters also has the potential to increase the number of alpha-particles that are delivered per targeted cell. For example, depending on the lamellarity, an 800 nm multilamellar vesicle will have approximately 21×10⁶ lipids per vesicle ¹³. As described in the methods, ²²⁵Ac-loaded 800 nm vesicles are formed by co-incubation of 25×10¹⁷ cholesterol-phosphatidyl choline complexes with a given activity A₀ of ²²⁵Ac in a 2 ml volume. Extrusion of this yields approximately 1.2∴10¹¹ vesicles, each containing at least 0.0334*A₀ (and two in ten, containing 2*0.0334*A₀) atoms of ²²⁵Ac when the encapsulation efficiency is 10%.

If for example 150 μCi are used, than each vesicle will contain a least 5 (and two in ten, will contain 10) atoms of ²²⁵Ac. Over a 30 to 40 day period, each ²²⁵Ac atom will yield 4 alpha particle emissions. Assuming, conservatively, that only 10 liposomes are bound to each tumor cell, each cell would get irradiated by more than 10*5*4=200 alpha-particles. Again, assuming conservatively that these decays occur on the surface, approximately ⅕ would traverse the nucleus (for a cell where the nuclear diameter is 80% of the cellular diameter). The resulting 40 nuclear traversals would assure sterilization of targeted cells. A secondary advantage of liposomal targeting is the ability to simultaneously coat the liposomes with antibodies that target different antigens, thereby reducing the possibility of not targeting a particular population of tumor cells, e.g., ones that do not express a particular antigen.

Routes of administration of immunolabeled MUVELs are dependent on the disease targeted and are well-known to those or ordinary skill in the art. Additionally, such an artisan would be skilled in determining dosage and use of therapeutic radionuclides. As such, it is appreciated that the means and methods of use of multivesicular liposomes for targeted radionuclide delivery is not limited by the instant disclosure.

The following examples are given for the purpose of illustrating various embodiments of the invention and are not meant to limit the present invention in any fashion.

### EXAMPLE 1

Determination of Actinium-225 Leakage From Unilamellar Liposomes and Loss Rate of Emitted Daughters

Analysis of Indium-111 Leakage From Liposomes

To optimize the liposomal formulation and evaluate potential leakage of radionuclides from liposome constructs, indium-111 was used in place of Ac-225. Indium-111 (In-111) is easily detected by gamma counting and both indium-111 and actinium-225 form tri-chloride complexes. Thus, In-111 serves as a test model for liposomal encapsulation of Ac-225. The method described by Hwang et al.^{?} was used with minor modifications.

Liposomes were isolated by Sephadex™ chromatography and loaded with ¹¹¹In by incubation for 1 h at room temperature with a loading solution consisting of 6-10 μl 6.9 mM oxine in oxine sulfate in deionized water with 200 μl 1.8% NaCl/20 mM sodium acetate, pH 5.5. To this acetate buffer, an equal volume of ¹¹¹InCl₃ in 3 mM HCl was added to make the final loading solution. Loading was terminated by passage through an AGIX ion-exchange column. The fractions corresponding to liposomes were pooled and stored at 4° C. and 37° C. for evaluation of indium leakage.

At different times after ion-exchange chromatography, aliquots of the pooled liposomal fractions were drawn, chromatographed by size exclusion (Sephadex™ column) separation and counted for radioactivity in a gamma counter. The counts in liposomal fractions were expressed as a fraction of the radioactivity aliquoted from the original pooled sample. In selected samples, prior incubation with DTPA was included to ensure that retention of ¹¹¹In in liposomal fractions was not the result of leakage and equilibration of ¹¹¹In between extra- and intra-liposomal ¹¹¹In in the pooled sample.

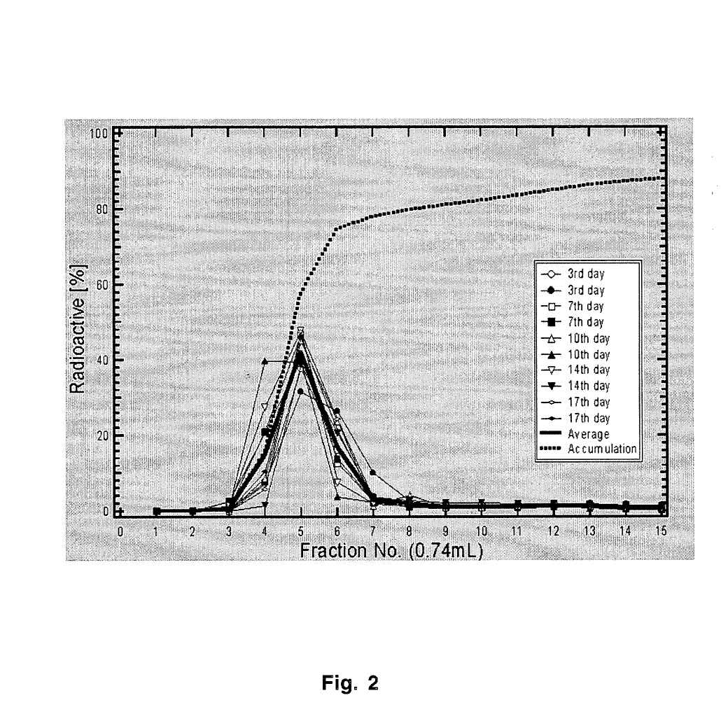

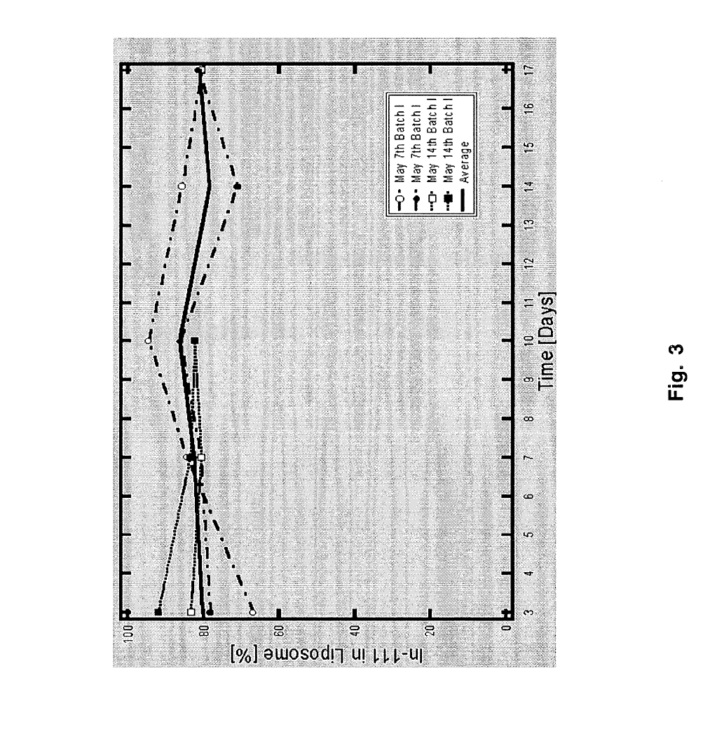

Following ion-exchange chromatography, approximately 20% of the radioactivity remains on the ion-exchange resin, and close to 80% in the liposomal fractions, yielding an encapsulation efficiency of approximately 80%. The radioactivity collected in each fraction after Sephadex™ column chromatography at different times after ¹¹¹In encapsulation is depicted in FIG. 2. The results show a generally time-invariant profile following size-exclusion separation. FIG. 3 depicts the retention of ¹¹¹In in liposomes as a function of time after loading. The fraction of ¹¹¹In retained within liposomes appears to remain constant at approximately 80% over a prolonged time-period, indicating that retention of Ac-225 within liposomes is possible.

Analysis of Actinium-225 Leakage From Liposomes

These same experiments were performed using Ac-225. In this case, gamma counting provided information regarding the presence of daughter radionuclides within the liposomes. Liposomes were incubated for 1 hr with Ac-225. To determine loss rate of daughter radionuclides from liposome-encapsulated Ac-225 activity, Ac-225 containing liposomes were separated from free Ac-225 by elution through a Sephadex™ column. The fractions corresponding to the liposomes, i.e., fractions 4-6, based on In-111 studies, were pooled and counted on a gamma counter using windows appropriate for detection of the Fr-221 and Bi-213 photopeaks, 218 and 440 keV, respectively. A 1-minute counting interval was selected and counting was performed overnight. Fr-221 counts were decay corrected to the start of the 1-min. counting period. At the end of separation and 10 to 20 h later (if possible), the column were also counted in a dose calibrator.

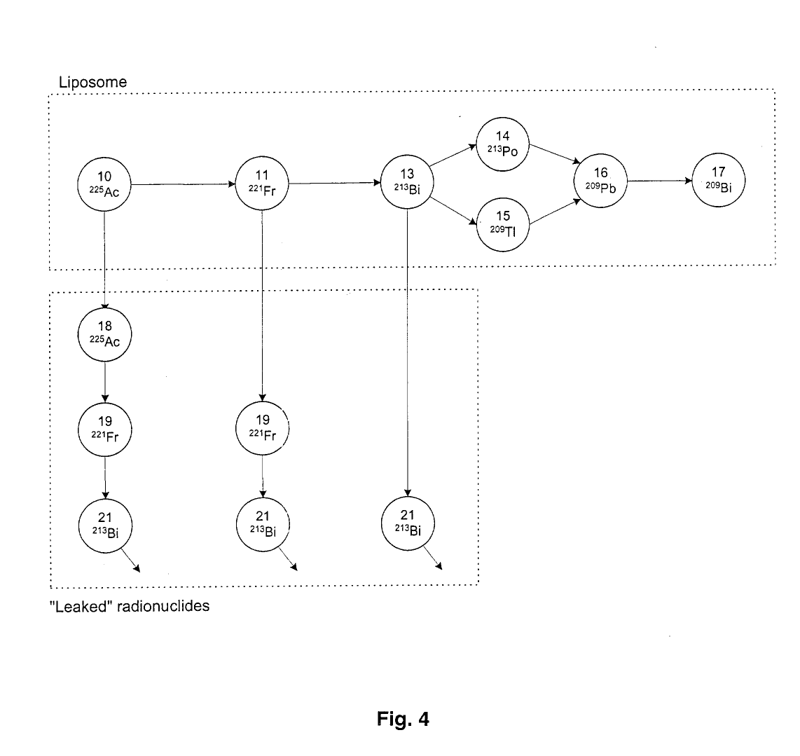

The results were analyzed using the model of Ac-225 radioactive decay shown in FIG. 4. Sub-compartments 10-17 correspond to decays that occur within liposomes. Loss of daughter or parent radionuclide is depicted by a “leak” rate from the liposome compartments to corresponding sub-compartments in the “leaked” radionuclides compartment. The transfer rate from each sub-compartment within liposomes to the extraliposomal compartment reflects a rate of loss from liposomes. This rate may be described in terms of a loss or clearance half-life, the amount of time required for Ω of a particular radionuclide to diffuse out of the liposome. Transfer rates within a compartment correspond to physical decay, whereas those that cross between the two compartments correspond to transfer of radionuclide from one to the other compartments. Since astatine-217, which has a 32 msec half-life is not resolved by this model, it is lumped with Fr-221.

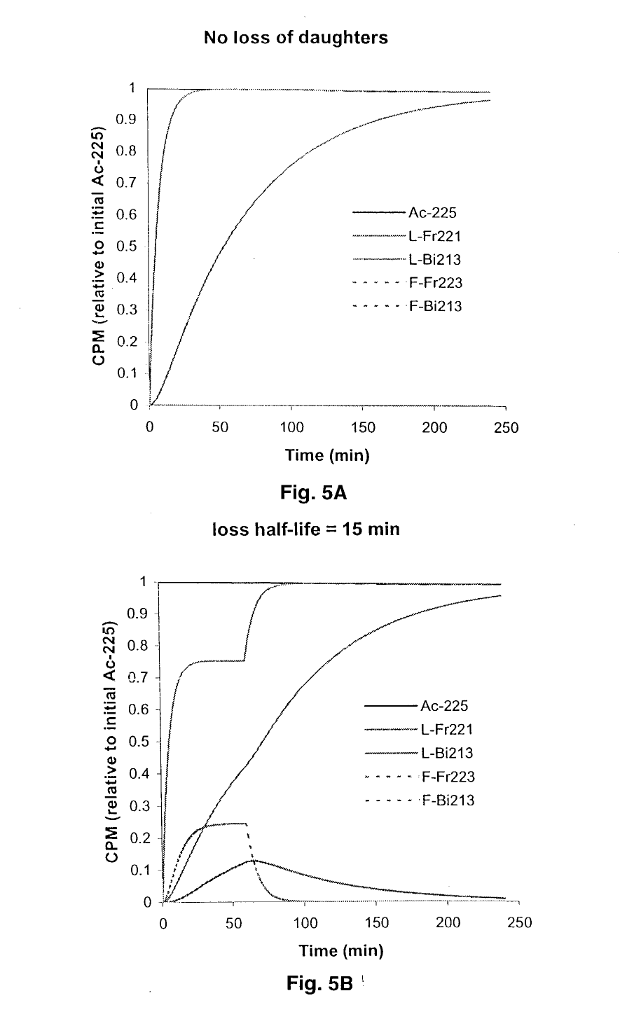

A sample simulation using this model is depicted in FIGS. 5A and 5B. FIG. 5A shows the results of a simulation in which complete retention of all radionuclides was assumed. Results are expressed relative to the initial activity of Ac-225 encapsulated by the liposomes. The first solid line shows the decay of Ac-225 while the other solid curves show the rise in daughter radioactivity within liposomes as equilibrium between the parent and daughters is reached. Note that because of its short, 5 min half-life, Fr-221 achieved equilibrium much more rapidly than Bi-213, which has a 45.6 min half-life. The two dotted lines, corresponding to Fr-221 or Bi-213 activity not within liposomes, are just visible at zero throughout the simulation duration.

FIG. 5B shows a simulation assuming loss of Fr-221 at a rate of 0.046 min⁻¹ which is equivalent to a loss half-life of 15 min. All other loss rates were set to zero. This means that Bi-213 generated within liposomes was assumed to remain there. Complete retention of Ac-225 was also assumed in this simulation.

As shown by the rise in both liposome-associated and free daughters (solid and dashed lines, respectively), equilibration and loss of daughters was allowed to occur during the 1-hr incubation period. After separation, and during counting of liposomal fractions, the distinction between free and liposome-encapsulated radioactivity was no longer possible. Therefore, the loss rate is turned off so that the solid curves correspond to total daughter radioactivity associated with the liposomal fractions (including daughters that were leaking out); the dashed curves reflect the physical decay of daughter radionuclides remaining in the column or in the liposome-free fractions. Although the latter could be assayed for radioactivity of Fr-221 and Bi-213, the method for assessing daughter loss rates was based upon counting the liposomal fractions.

Analysis of Daughter Loss From Unilamellar Liposomes Using Different Loss Rates

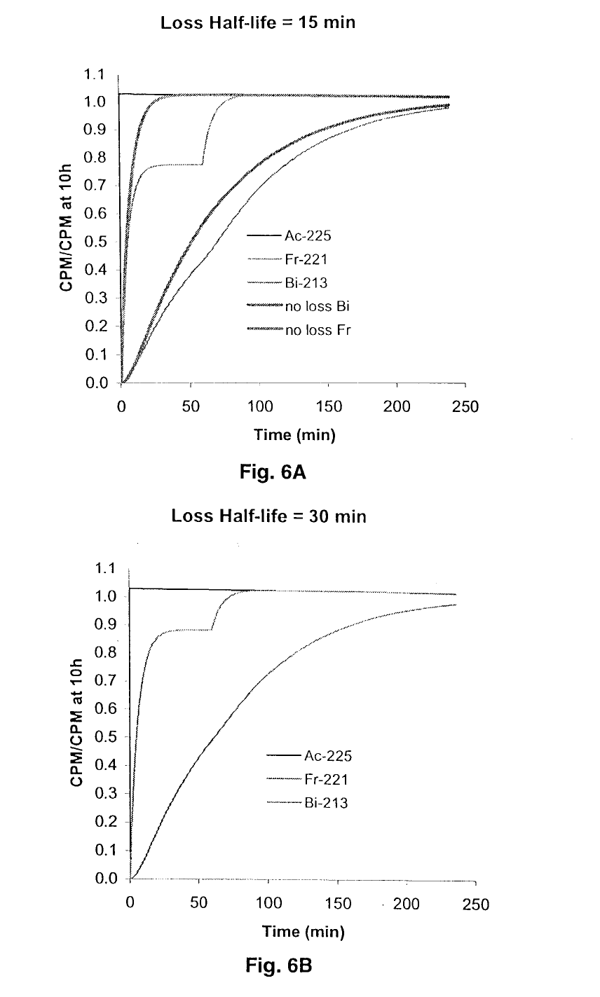

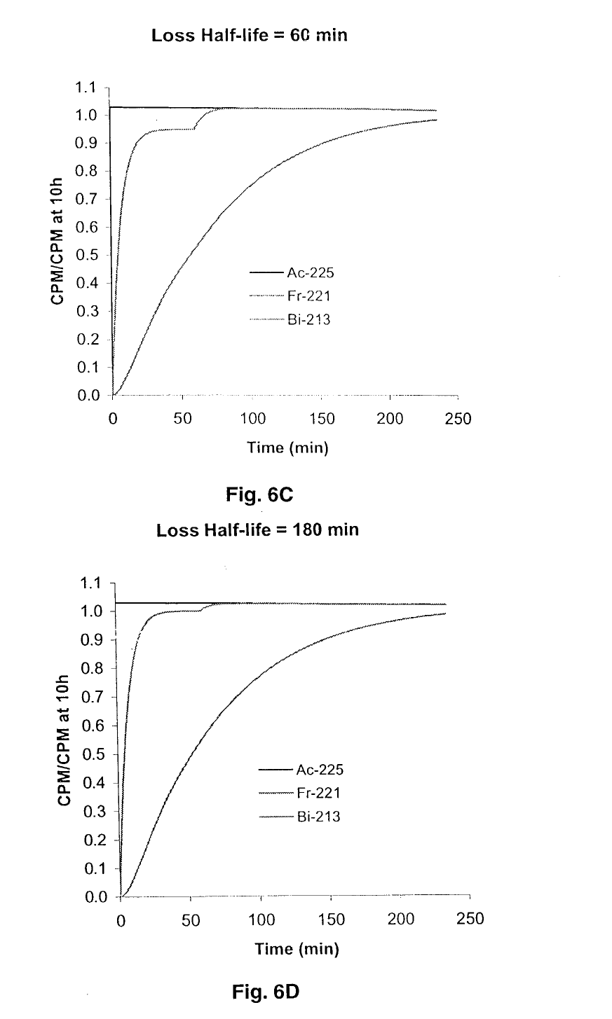

FIGS. 6A-6D depict simulations obtained using four different loss rates. The curves associated with counting of liposomal fractions are shown without the corresponding curves for free daughters collected on the column or in the liposome-free fractions. All other conditions are as described above. The CPM values are normalized by dividing by CPM expected after 10 h (i.e., at equilibrium).

In FIG. 6A where the loss half-life is modeled at 15 min the curves corresponding to a simulation that did not include loss of Fr are shown for comparison with the simulation that included loss. The level of Fr-221 and Bi-213 activity immediately after separation was sensitive to the loss half-life of Fr-221. This is clearly evident for Fr-221 since the loss rate impacted the equilibrium level (plateau) that was reached. It was less evident for Bi-213 since it does not reach equilibrium in 1 h. Once the separation had occurred, i.e., after 60 minutes, the distinction between free and liposome-associated daughter activity was lost over time as the daughters reached equilibrium with the parent. Correspondingly, as the measurement of daughter activity was delayed relative to the time of separation, the ability to distinguish the different loss rates is also reduced.

Relationship Between Loss Rate and Daughter Activity

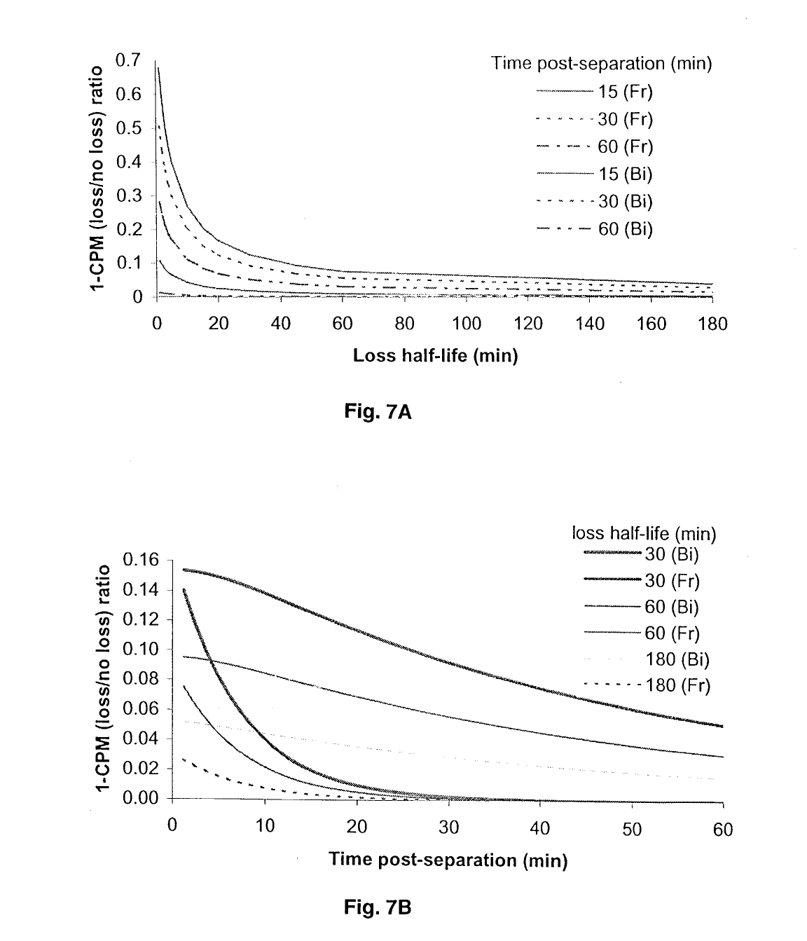

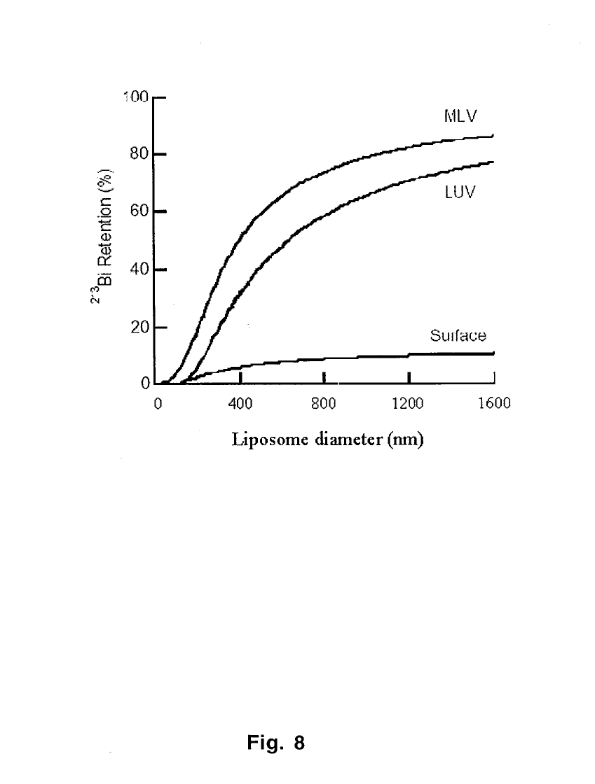

The relationship between loss rate and the levels of daughter activity at different measurement times after liposome separation is shown more directly in FIGS. 7A-7B. FIG. 7A shows the normalized count rate ratio, as presented in FIGS. 6A-6D, of each daughter divided by the corresponding count rate ratio assuming no loss of Fr-221 and then subtracted from one to yield curves that approach zero over time. Results are plotted against different loss half-lives and for measurement times of 15, 30 and 60 minutes after separation. If counting is started immediately after separation and carried out overnight, these data are available.

FIG. 7B is a different representation of the data used to generate FIG. 7A. Loss rate sensitivity is plotted against the time post-separation at which the liposomal fractions are counted. Curves are provided for three different loss half-lives: 30, 60 and 180 min.

Determination of the Loss Rate That Yields a Negligible Absorbed Dose to Kidneys From Bi-213

To determine the loss rate that yields a negligible absorbed dose to kidneys from Bi-213, a 5 day biological half-life of the Ac-225 construct is assumed and a 25 d simulation is plotted. Subsequently, the Bi-213 released for different Fr-221 release rates is integrated and used to perform rough estimate of dosimetry. 25 d=36000 min=simulation time 5 d half-life=9.627e-5

### EXAMPLE 2

Determination of Liposomal Size to Retain Actinium-225 Daughters Theoretical Model

The nuclear recoil distances of the alpha decays of the actinium daughters (francium, astatine, and bismuth) are not well established in aqueous media, but estimates to within 10-20% can be made¹⁴. Using a standard computer model (SRIM, Stopping and Range of Ions in Matter, James Ziegler, http://www.srim.org/), the range of these recoils can be estimated as 81.7, 86.5, 94.7 nm. Since these differ by less than the experimental uncertainties (both in the recoil range estimates and in our experimental measurements), a single recoil distance was used to simplify further calculations. Three models are demonstrated in FIG. 8.

The first is the “LUV”, or large unilamellar vesicle model, in which it is assumed that each entrapped radionuclide is distributed uniformly within the aqueous volume of a vesicle. This is shown in FIG. 8 as the theoretical line “LUV” which is calculated assuming a recoil distance of 87.6 nm, the average recoil distance in water calculated using SRIM. Larger vesicles are likely to be multilamellar ¹⁵. This may prevent the free diffusion of daughter nuclides within the vesicle.

In the absence of diffusion the loss of bismuth is better modeled by three recoil events. This calculation shows that a slightly higher retention probability should be achievable in multilamellar systems. The result is shown in FIG. 8 as the line marked MLV. The result for three successive “surface” disintegrations is also shown in FIG. 8. The maximum bismuth retention approaches ½³=12.5% for large vesicles, as expected. Half the recoils from the surface of an infinitely large vesicle would result in daughter ejection. Clearly, if even a fraction of the daughters of actinium (or if actinium itself) associates with the vesicle membrane, the bismuth retention will be significantly reduced. These models suggest that 700 to 800 nm-diameter liposomes are required to achieve adequate, i.e., >60%, bismuth retention.

Preparation of Unilamellar and Large Unilamellar Vesicles by the Extended Hydration Method

Various sizes of liposomes may be prepared by extrusion through nuclear track-etch membranes with differing pore sizes. Larger pores are known to produce liposome populations with larger mean diameters ¹⁵. It is contemplated that the liposome diameter may be larger or smaller than the pore diameter, possibly depending on variables such as lipid composition, and extrusion pressure or rate. Phospholipid hydration strongly influences the achievable liposome size. In addition, different compositions exhibit variable stability over time depending on liposome size.

Stable, PEGylated liposomes of 100 nm, 400 nm and 800 nm diameters are prepared. A mixture of phosphatidyl choline and cholesterol (1:1 mole ratio) and PEG-labeled lipid (6% total lipid) is dried in a rotary evaporator. For stability measurements, the lipids are resuspended in calcein solution (55 mM calcein in phosphate buffered saline (PBS), pH=7.4). For actinium passive entrapment, the lipids are resuspended in PBS containing chelated actinium complexes (DOTA-Ac and DTPA). The lipid suspension is annealled at 55° C. for 2 hours 16. To make vesicles the lipid suspension is taken through twenty-one cycles of extrusion (LiposoFast, Avestin) through two stacked polycarbonate filters (100 nm, 400 nm, 800 nm). Unentrapped contents are then removed by size exclusion chromatography (SEC) in a Sephadex G-50 packed 1×10 cm column, eluted with an isotonic buffer. Unentrapped small liposomes are removed by SEC in a Sephacryl S-1000 packed column. Ascorbic acid (8 mM) is coentrapped to minimize lipid oxidation due to radiation. In addition, other radioprotective agents may also be incorporated into the liposome^{17, 18, 19}.

Dynamic Light Scattering

The liposome size is verified by dynamic-light scattering. Dynamic light scattering (DLS) of vesicle suspensions is studied with an N4 Plus autocorrelator (Beckman-Coulter), equipped with a 632.8 nm He—Ne laser light source. Scattering is detected at 15.7°, 23.0°, 30.2°, and 62.6°. Particle size distributions are calculated from autocorrelation data analysis by CONTIN²⁰. All buffer solutions are filtered with 0.22 μm filters just prior to vesicle preparation. DLS will be carried out in collaboration with Prof. Teraoka at Polytechnic University (Brooklyn, N.Y.).

TEM Measurements:

To observe the size and external morphology of liposomes, a JOEL transmission electron microscope (TEM) may be used at 80 kV following the negative staining method. Vesicle suspensions are added dropwise to a 400-mesh copper grid coated with Formvar®. After allowing for vesicle adhesion excess sample is removed with filter paper. Staining is done with isotonic uranyl acetate solution (2%) in PBS. The internal structure of the multivesicular vesicles will be examined by cryo-TEM in collaboration with Mr. Macaluso at the Analytical Imaging Facility, Albert Einstein College of Medicine (Bronx, N.Y.).

Stability of LUV Liposomes

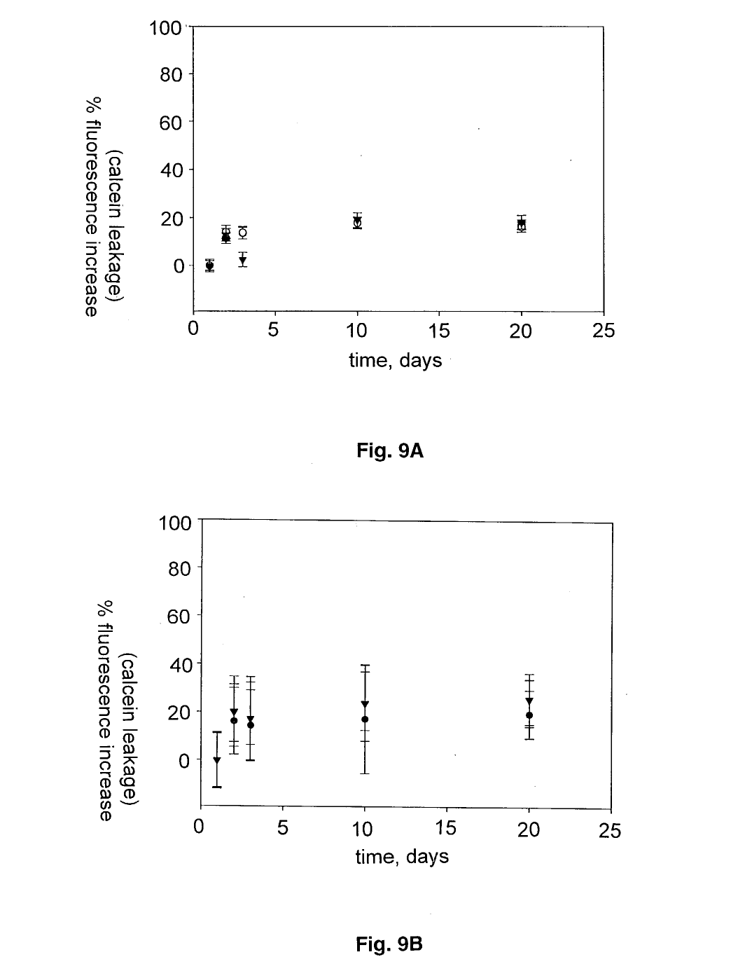

The stability of two different liposome compositions, zwitterionic and positively charged, was tested with entrapped calcein (a fluorophore at self-quenching concentrations). FIGS. 9A-9B show the percentage of fluorescence increase due to calcein leakage over time. After the first 24 hours a 20% fluorescence increase was measured for all vesicles (possibly due to differences in osmotic pressure across the liposomal membrane). Beyond this point all vesicles were stable for over 20 days.

Radionuclide Retention in ULV and LUV

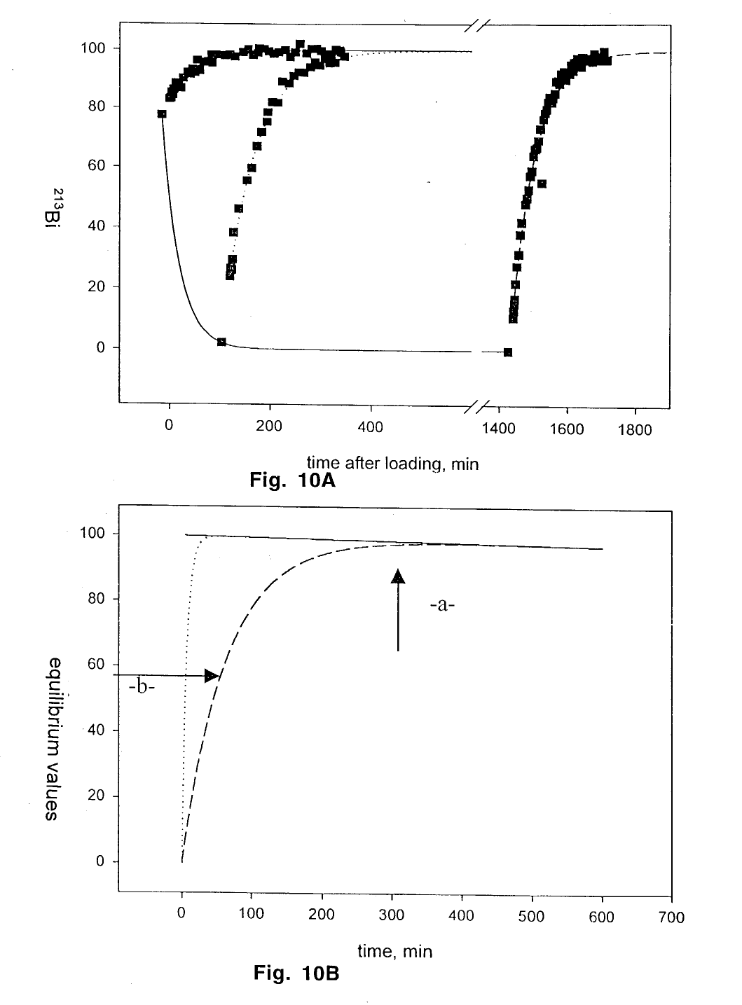

A number of studies examining the radionuclide retention of liposomes have been carried out. Results are summarized below. An experiment with 100 nm (in diameter) liposomes is shown in FIG. 10A. Vesicles were prepared as described in the methods. Actinium was passively entrapped in the vesicles during extrusion, and then unentrapped actinium was separated using size-exclusion chromatography. As shown in FIG. 10 B if ²¹³Bi is not leaking then the ²¹³Bi activity concentration as measured by gamma counting would be the same over several hours of measurement since it would be at equilibrium with 225Ac (10d half-life).

In the experiment shown in FIG. 10A the vesicles were rechromatographed after 24 hours, and the ²¹³Bi gamma emissions from the vesicle fraction were measured. The low initial ²¹³Bi activity shows that bismuth has been largely lost from the vesicles. Had bismuth-213 been retained, the radioactivity of the vesicle fraction would have been at steady state with ²²⁵Ac,i.e., at ˜100%. In this newly-separated vesicle fraction, the bismuth activity rises as ongoing actinium decay gradually brings the entire fraction into steady state.

Measurements of Steady State

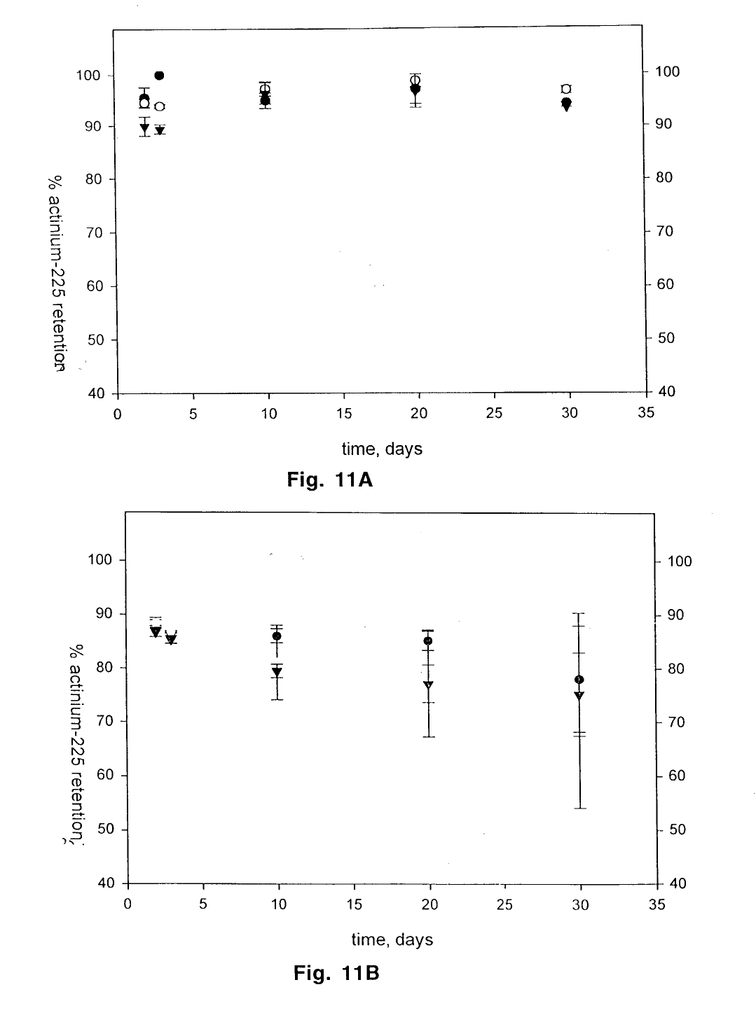

²¹³Bi activity in vesicle fractions separated from the parent vesicle population at different times allow for an estimate of the stability of actinium entrapment. FIGS. 11 A-11B depict ²²⁵Ac retention measurements for 100, 400 and 800 nm liposomes vesicles produced by extended hydration for both liposome compositions over a period of 30 days. FIG. 11A shows that, independent of time, the activity recovered in the zwitterionic vesicle fraction is >88% of ideal. The loss may be caused by the chromatographic separation itself. In FIG. 11B actinium-225 retention decreases over time in the positively charged liposomes, but even after 30 days the retention is more than 54%.

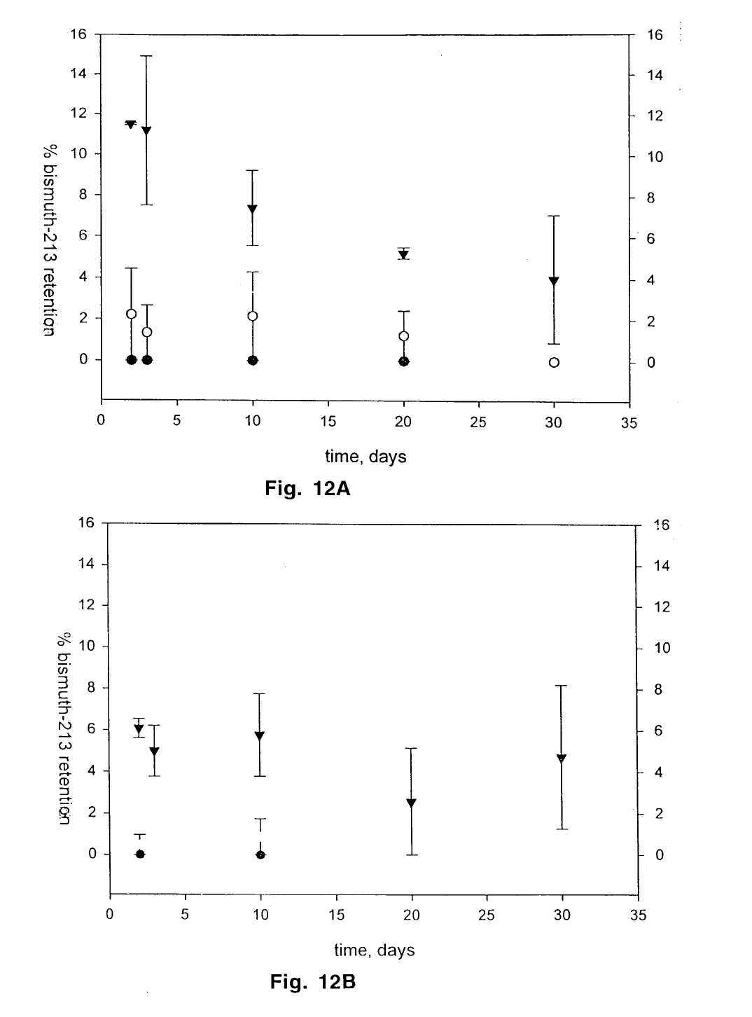

The retention of ²¹³Bi from vesicles was then studied as a function of liposome size and composition. In FIGS. 12A-12B for each liposome population, measurements of ²¹³Bi activity were made following rechromatography at various time points. For large liposomes, retention is much less than theoretically predicted. The initial drop in FIG. 12A for zwitterionic 800 nm liposomes (▾) may be explained by slower approach and localization of the actinium-chelate complex to the liposomal membrane. For both liposome compositions and all sizes examined, the values of bismuth retention are consistent with the theoretical results for localization of the radionuclides on the liposome surface (the membrane).

### EXAMPLE 3

Multivesicular Liposomes (MUVELs) for Enhanced Retention of Actinium-225 and its Daughters

Basic Structure of MUVELs

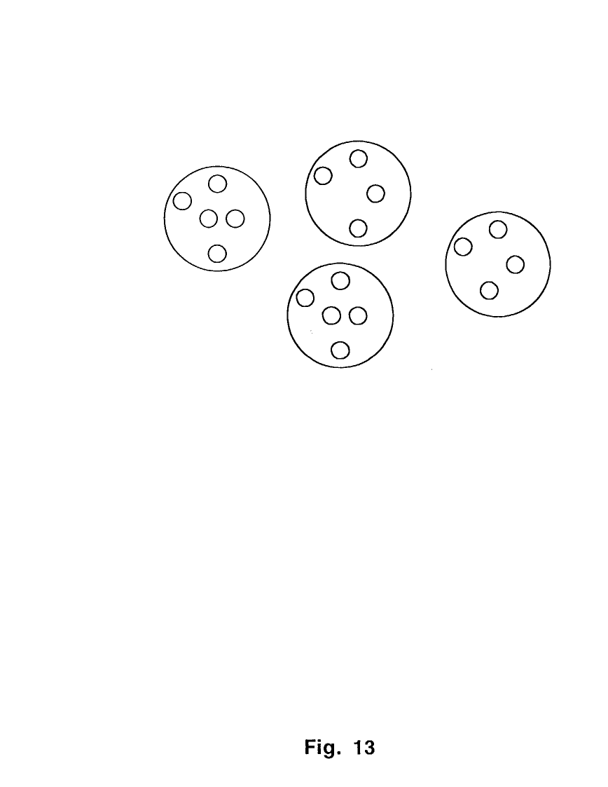

As determined by the theoretical model described in Example 2 and depicted in FIG. 8, enhanced bismuth retention can only be achieved when the radionuclides are localized within the liposomes. By loading actininium-225 into small liposomes and entrapping these into a large unilamellar liposome having a diameter of about 700-800 nm enhanced bismuth-213 retention is achieved. This liposomal design (FIG. 13) allows for excess membrane within the larger liposomes and hence radionuclide localization within the core of the larger liposomes.

Separation Quality Between Large and Small Liposomes

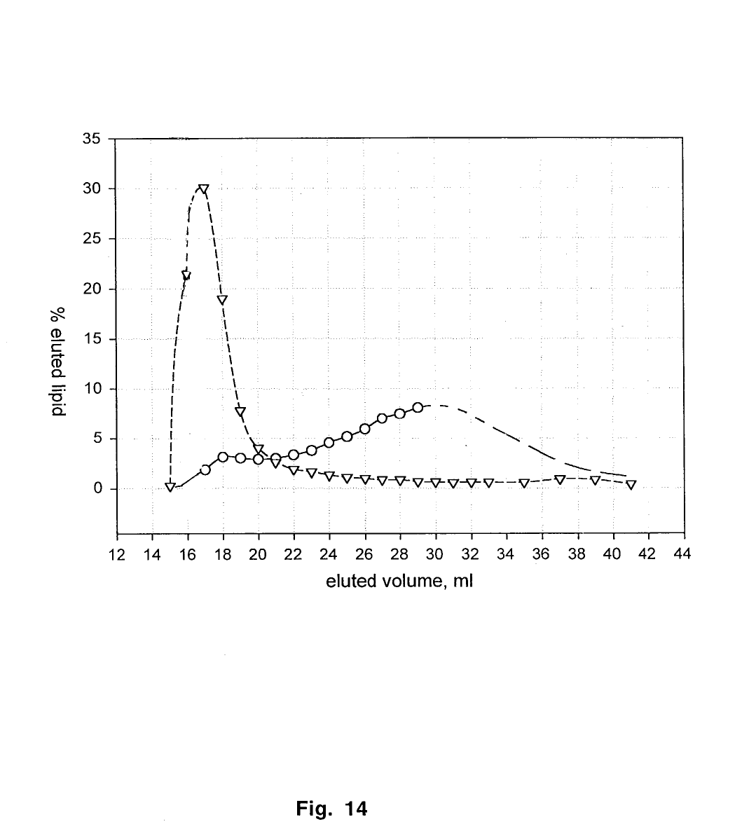

As shown in FIG. 14, to evaluate the separation quality between large (800 nm filter diameter) and small (sonicated) liposomes by S-1000 column, the elution profiles of liposomes of the above sizes were obtained in parallel measurements. Liposomes were labeled with rhodamine lipids (0.5% total lipid) to enable liposome detection. Separation is relatively good but not satisfactory for the purpose of our application. Further purification is obtained with affinity chromatography.

Preparation of Small Liposomes by Extended Hydration Method

To make small liposomes the lipid suspension as prepared in Example 2 is sonicated in a bath sonicator for thirty minutes and is then taken through twenty-one cycles of extrusion (LiposoFast, Avestin) through two stacked polycarbonate filters (100 nm). Unentrapped contents were then removed by size exclusion chromatography (SEC) in a Sephadex G-50 packed 1×10 cm column, eluted with an isotonic buffer (PBS).

Preparation of Multivesicular Liposomes

A mixture of phosphatidyl choline and cholesterol (1:1 mole ratio), and PEG-labeled lipid (6% total lipid) in CHCl₃ were dried in a rotary evaporator at 55° C. For coentrapment of smaller liposomes, the lipids were resuspended in the solution of sonicated liposomes. The lipid suspension was then annealled to 55° C. for 2 hours. To make large liposomes the lipid suspension was then taken through twenty-one cycles of extrusion (LiposoFast, Avestin) through two stacked polycarbonate filters (800 nm). Unentrapped small liposomes were removed by size exclusion chromatography (SEC) in a Sephacryl S-1000 packed column. Larger liposomes whose outer membrane consists partially or entirely of the membrane of small liposomes were removed by a streptavidin labeled Sepharose high-trap affinity column (HiTrap Streptavidin HP, Amersham Biosciences). Ascorbic acid (8 mM) was coentrapped to minimize lipid oxidation due to radiation.

Verification of Small Vesicle Entrapment

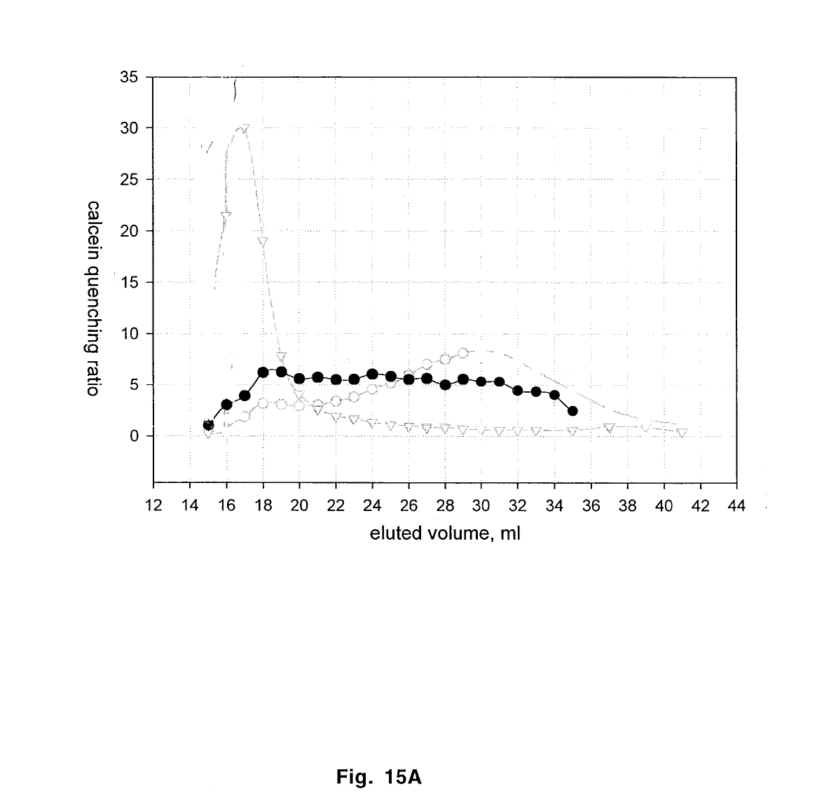

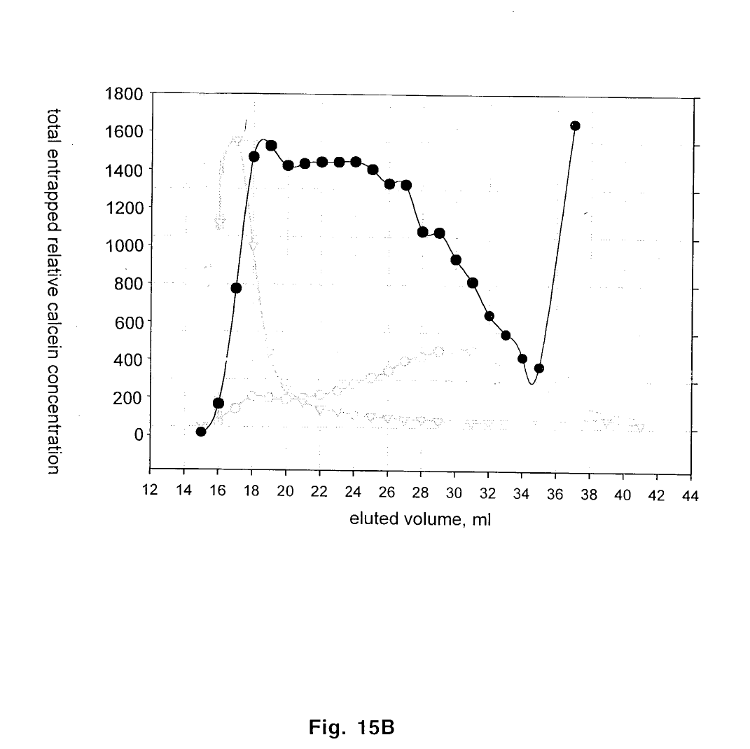

To verify the presence of entrapped small vesicles in large liposomes, multivesicular liposomes were prepared according to methods with small liposomes containing self-quenching amounts of calcein. MUVELs were separated from unentrapped small liposomes by size exclusion chromatography (S-1000). The fluorescence intensity of each fraction was measured before and after triton X-100 addition; the ratio of the fluorescence signals is shown in FIG. 15A. Quenching of calcein in the fractions 16-17-18 where the large liposomes mainly elute (FIG. 14) implies the presence of entrapped small liposomes in the large 800 nm liposomes. Further structural characterization will include cryo-TEM (transmission electron microscopy). This method allows for direct tomographic imaging of the lamellae at resolution higher than 100 nm. In addition, as shown in FIG. 15B, the total concentration of calcein (after triton x-100 addition) per MUVEL fraction (16-17-18) is indicative of the amount of small liposomes entrapped.

Stability of MUVELs

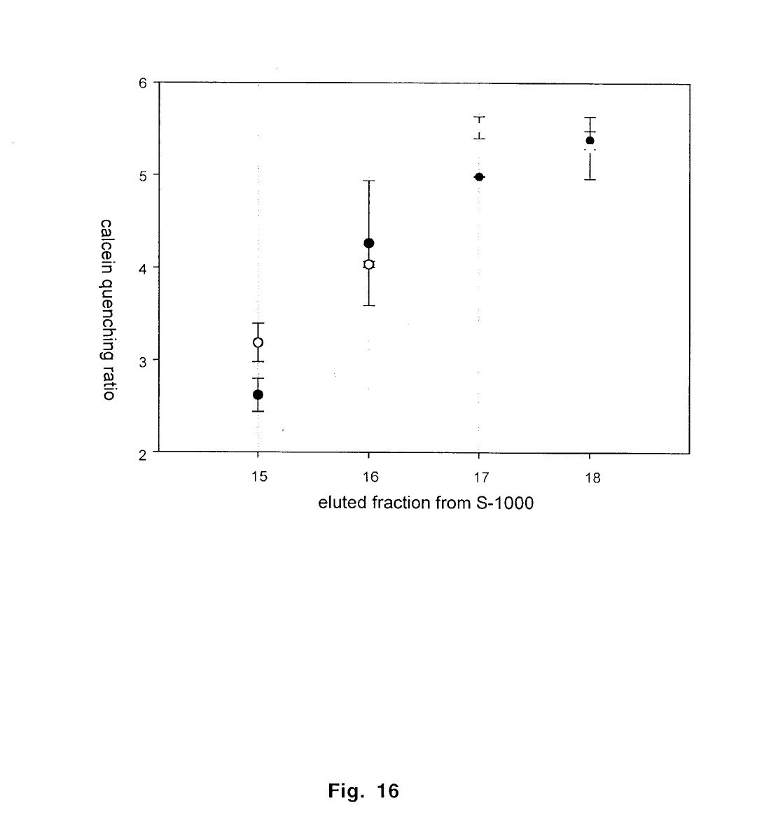

To study the stability of MUVELs, the quenching ratio of each fraction was followed in time (FIG. 16). After four days (closed symbols) the relative quenching ratios of the fractions shown are not significantly different. The calcein quenching ratio of small liposomes that have not been encapsulated into the MUVELs was stable during the above time period and of higher value: 9.6±1.2, indicating that calcein leakage has occurred in the encapsulated small liposomes.

In addition, during the MUVEL preparation at the second step of annealing, various liposomal structures may be formed whose external membrane originates from small liposomes, i.e., unentrapped fused small liposomes that formed larger structures or small liposomes fused with large liposomes. These structural forms, independently of their size, should be removed from the MUVEL suspension. Radionuclides were shown to localize on the membrane surface, and they will bind to the membrane of small liposomes. Thus, any part of these membranes should be removed from the surface of the final large liposomal structures, otherwise the daughter retention will decrease dramatically.

To separate these forms from MUVEL, the membrane of small liposomes was labeled with biotin. Use of streptavidin coated affinity column removes all of the above structures. It is contemplated that to minimize these fusion events, different liposome compositions can be used for the sonicated small liposomes which have transition temperatures significantly higher than 55° C. that is used in the preparation protocol.

Radionuclide Retention of Liposomes

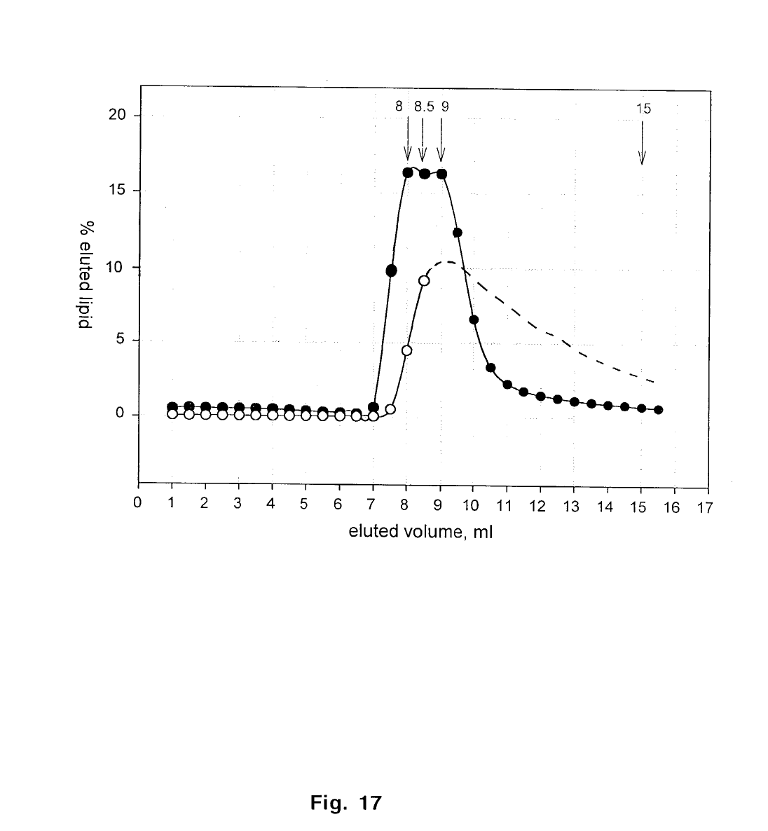

Small sonicated liposomes were prepared as described in the methods. Actinium was passively entrapped in small liposomes and unentrapped actinium was separated using size-exclusion chromatography (G-50). Small liposomes were then entrapped in large (800 nm, filter diameter) liposomes. MUVELs were separated by S-1000 (FIG. 17) and each fraction was purified by streptavidin-labeled affinity column. Radionuclide retention results are shown for the fractions indicated with arrows (8, 8.5, 9, 15).

Actinium and Bismuth Retention Measurements

Actinium-225 decays to the following R-emitting radionuclides (respective half-lives shown): ²²¹Fr (4.9 min), ²¹⁷At (32 msec), and ²¹³Bi (45.6 min). To experimentally test the retention of actinium and its daughters by the vesicles, the γ emissions of francium-221 and bismuth-213 were measured; α-emissions are difficult to detect. Under steady state conditions, the decay rate of each species in the decay chain must be equal; thus, at steady state, the decay rate of either francium or bismuth can be used to determine the actinium concentration. Measurements of the kinetics of ²¹³Bi activity in vesicle fractions separated from the parent vesicle population at different times allow for estimation of the stability of bismuth and actinium entrapment. For each vesicle population, measurements of ²¹³Bi activity were made following rechromatography at various time points.

Retention of Actinium-225 and Bismuth-213 in MUVELs

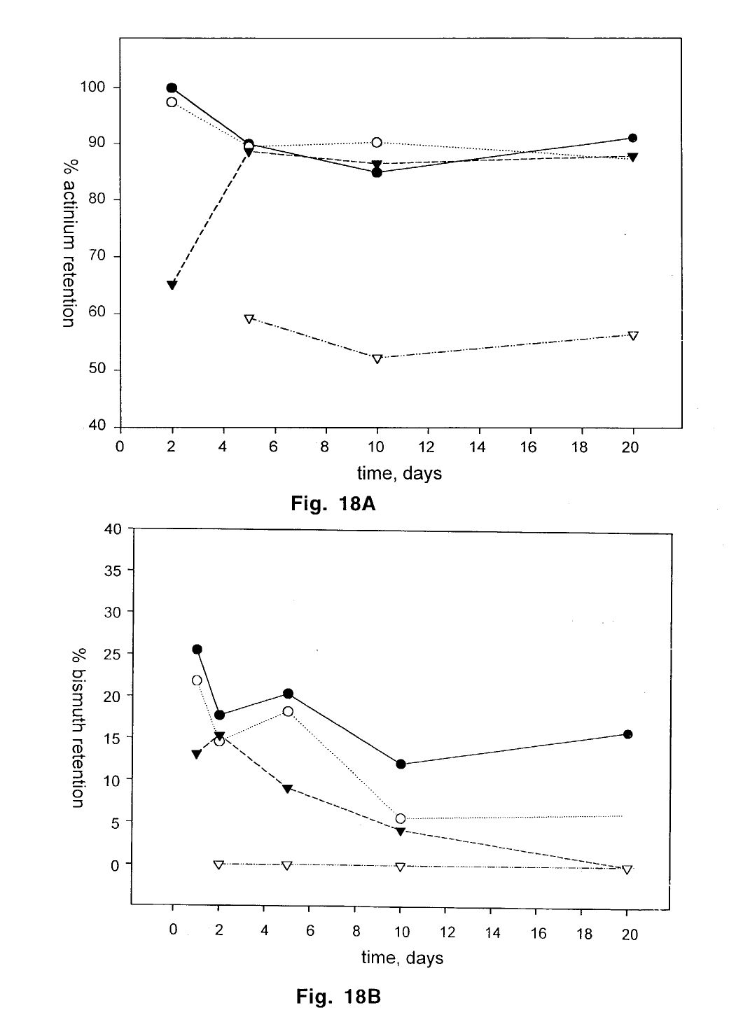

In FIG. 18A the retention of actinium-225 was then measured for each fraction over the period of 20 days. Fractions 8, 8.5 and 9 have shown satisfactory actinium retention. Fraction 15 (which consists mainly of unentrapped small liposomes) is characterized by actinium leakage.

As shown in FIG. 18B bismuth-213 retention is significantly improved compared to large 800 nm liposomes without encapsulated vesicles (5% retention). In particular, fraction 8 was shown to have the highest bismuth retention (16% after 20 days). The decrease with time of bismuth retention may possibly be explained by leakage of actinium from the entrapped small liposomes (in FIG. 18A, fraction 15 shows actinium leakage) into the encapsulated aqueous compartment of the large liposomes. This would result in localization of the escaped radionuclides on the membrane surface of the large liposomes, and thus decrease of bismuth retention. Fraction 15, which is consisted mainly of unentrapped small liposomes, does not retain bismuth.

### EXAMPLE 4

Targeted Delivery of Immunolabeled MUVELs Preparation of Immunoliposomes

Antibodies are attached at the terminal end of the PEG-chains of the vesicles; this geometry provides exposed antibody molecules that protrude from the vesicle for unhindered antigen recognition. In particular, vesicles are prepared with 1-3% mole carboxy-terminated PEG-lipids. The carboxy terminus is modified to an amine reactive NHS (N-Hydroxysuccinimide) ester by mixing the NHS and the dehydrating agent EDC (1-ethyl-3-(3-dimethylaminopropyl)carbodiimide hydrochloride) in the vesicle suspension. The vesicles are immunolabeled by reaction of the modified carboxy-termini with the epsilon amine groups of lysines on antibodies²¹. Immunoliposomes are then separated from the unreacted antibodies by a Sepharose 4B size exclusion column preequilibrated with PBS ²².

Murine Model

In more than 40 mouse experiments, intraperitoneal injection of 5×10⁶ previously passaged SKOV3 cells in 4-6 week old female Balb/c nu/nu mice has yielded 100% tumor take. By three weeks, discrete, mm-sized tumor nodules are observed around the spleen; in many cases such nodules are also found on the surface of the GI tract and liver. By four weeks discrete tumor nodules are replaced by contiguous masses of tumor around the spleen, GI tract and liver. At this time ascites fluid is also observed.

Biodistribution of Herceptin-Labeled Immunoliposomes

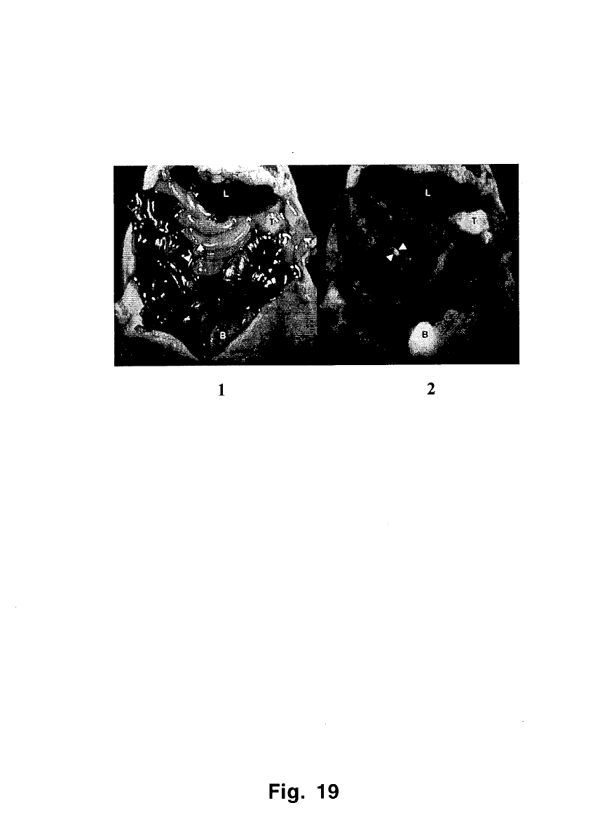

MicroPET and MRI-based pre-clinical biodistribution and localization data have been obtained using ⁸⁶Y-Herceptin (HER) antibody (anti-HER2/neu) against ovarian carcinoma ^{24,25}. Radiolabeled Herceptin Ab was shown to localize to sites of disease with minimal normal organ uptake. Optical images of FITC-Herceptin have also been obtained to provide a high resolution assessment of Herceptin targeting as shown in FIG. 19. This methodology is also used to evaluate tumor targeting of liposomes immunolabeled with Herceptin.

Efficacy of Immunoliposomes Against IP-Disseminated Ovarian Carcinoma

To evaluate the fate of immunoliposomes, e.g., Herceptin-labeled immunoliposomes, in vivo, both the aqueous and the lipid membrane compartments are labeled with fluorescent markers. The aqueous compartment, i.e., contents, is labeled by entrapping calcein as described in Example 3. The membrane is labeled with NBD and rhodamine-tagged lipids to allow for fluorescence energy transfer. Thus, both the entrapped calcein and rhodamine are excited with the same laser light source. This enables simultaneous and independent detection of both compartments using different filters, making it possible to detect endocytosis/fusion of vesicles as well as vesicle integrity by multi-color fluorescence imaging and microsopy.

The following references were cited herein:

1. Humm J. A microdosimetric model of astatine-211 labeled antibodies for radioimmunotherapy. Int J Radiat Oncol Biol Phys 1987;13:1767-73.

2. Humm J, Chin L. A model of cell inactivation by alpha-particle internal emitters. Radiat Res 1993;134:143-50.

3. Macklis R, Kinsey B, Kassis A, Ferrara J, Archer R, et al. Radioimmunotherapy with alpha-particle-emitting immunoconjugates. Science 1988;240:1024-26.

4. Jurcic J, McDevitt M, Sgouros G, Ballangrud A, Finn R, et al. Targeted alpha-particle therapy for myeloid leukemias: A phase I trial of bismuth-213-HuM195 (anti-CD33). Blood 1997;90:2245.

5. Sgouros G, Ballangrud A, Jurcic J, McDevitt M, Humm J, et al. Pharmacokinetics and dosimetry of an alpha-particle emitter labeled antibody: 213Bi-HuM195 (anti-CD33) in patients with leukemia. J Nucl Med 1999;40:1935-46.

6. Zalutsky M, Cokgor I, Akabani G, Friedman H, Coleman R, et al. Phase I trial of alpha-particle-emitting astatine-211 labeled chimeric anti-tenascin antibody in recurrent malignant glioma patients. Proceedings of The American Association for Cancer Research 2000;41:544.

7. Behr T, Behe M, Stabin M, Wehrmann E, Apostolidis C, et al. High-linear energy transfer (LET) alpha versus low-LET beta emitters in radioimmunotherapy of solid tumors: therapeutic efficacy and dose- limiting toxicity of 213Bi- versus 90Y-labeled CO17-1A Fab′ fragments in a human colonic cancer model. Cancer Res 1999;59:2635-43.

8. Behr T, Sgouros G, Stabin M, Behe M, Angerstein C, et al. Studies on the red marrow dosimetry in radioimmunotherapy: an experimental investigation of factors influencing the radiation-induced myelotoxicity in therapy with beta-, Auger/conversion electron-, or alpha-emitters. Clin Cancer Res 1999;5:3031s-43s.

9. Kennel S J, Mirzadeh S. Vascular targeted radioimmunotherapy with 213Bi—an alpha-particle emitter. Nucl Med Biol 1998;25:241-46.

10. Zalutsky M, McLendon R, Garg P, Archer G, Schuster J, et al. Radioimmunotherapy of neoplastic meningitis in rats using an alpha-particle-emitting immunoconjugate. Cancer Res 1994;54:4719-25.

11. McDevitt M, Ma D, Lai L, Simon J, Borchardt P, et al. Tumor therapy with targeted atomic nanogenerators. Science 2001 ;294(1537-1540).

12. Kennel S J, Brechbiel M W, Milenic D E, Schlom J, Mirzadeh S. Actinium-225 conjugates of MAb CC49 and humanized delta CH2CC49. Cancer Biother Radiopharm 2002;17:219-31.

13. Brzozowska I, Figaszewski Z A. The equilibrium of phosphatidylcholine-cholesterol in monolayers at the air/water interface. Colloids Surf B 2002;23:51-58.

14. Ziegler J. Stopping and range of ions in matter (SRIM96). Yorktown, N.Y.: IBM, 1996.

15. Mayer L, Hope M, Cullis P. Vesicles of variable sizes produced by a rapid extrusion procedure. Biochim Biophys Acta 1986;858:161-68.

16. Castile J D, Taylor K M G. Factors affecting the size distribution of liposomes produced by freeze-thaw extrusion. Int J Pharm 1999;188:87-95.

17. Agarwal S, Chatterjee S N. Peroxidation of the dried thin film of lipid by high-energy alpha particles from a cyclotron. Radiat Res 1984;100:257-63.

18. Samuni A M, Barenholz Y, Crommelin D J A, Zuidam N J. g-irradiation damage to liposomes differing in composition and their protection by nitroxides. Free Rad Biol Med 1997;23:972-79.

19. Stensrud G, Redford K, Smistad G, Karlsen J. Effects of gamma irradiation on solid and lyophilised phospholipids. Rad Phys Chem 1999;56:611-22.

20. Provencher S W. A constrained regularization method for inverting data represented by linear algebraic or integral equations. Comput Phys Commun 1982;27:213-27.

21. Maruyama K, Takizawa T, Yuda T, Kennel S J, Huang L, Iwatsuru M. Targetability of novel immunoliposomes modified with amphipathic poly(ethylene glycol)s conjugated at their distal terminals to monoclonal antibodies. Biochim Biophys Acta 1995; 1234:74-80.

22. Kirpotin D B, Park J W, Hong K, Zalipsky S, Li W -L, Carter P, et al. Sterically stabilized anti-HER2 immunoliposomes: design and targeting to human breast cancer cells in vitro. Biochemistry 1997;36:66-75.

23. Rippe B, Rosengren B I, Venturoli D. The peritoneal microcirculation in peritoneal dialysis. Microcirculation 2001;8(5):303-20.

24. Palm S, Enmon R, Matei C, Kolbert K, Borchardt P. Pharmacokinetics of 86Y-Herceptin in an ovarian carcinoma model: correlative microPET and MR imaging. J Nucl Med 2002;43:153P.

25. Palm S, Enmon R, Matei C, Kolbert K, Xu S, Pellegrini V, et al. Pharmacokinetics and biodistribution of 86Y-Herceptin for 90Y dosimetry in an ovarian carcinoma model: correlative microPET and MR imaging. submitted to Cancer Res.

26. Hwang, K. J., et al. Encapsulation, with High Efficiency, of Radioactive Metal Ions in Liposomes. Biochim Biophys Acta 716.1 (1982): 101-9.

Any patents or publications mentioned in this specification are indicative of the levels of those skilled in the art to which the invention pertains. These patents and publications are herein incorporated by reference to the same extent as if each individual publication specifically and individually was incorporated by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

What is claimed is:

1. A method of preventing the systemic release of radioactive decay intermediates upon administration of an alpha particle-emitting radionuclide to an individual, comprising the steps of:
incorporating said radionuclide into large liposomes, said liposomes having a diameter sufficient to retain said radioactive decay intermediates; and
administering said large liposomes to said individual, wherein said radioactive decay intermediates remain sequestered within said large liposomes.

2. The method of claim 1, further comprising the step of:
entrapping said radionuclide within a smaller liposomal vesicle prior to incorporating said radionuclide contained therein into the aqueous phase of said larger liposome.

3. The method of claim 1, further comprising the step of:
coating outer membrane surfaces of said large liposomes with molecules which preferentially associate with a specific target cell thereby increasing specificity of said large liposomes to said target cell.

4. The method of claim 3, wherein said molecules are specific to tumor cells.

5. The method of claim 3, wherein said molecules are antibodies, peptides, engineered molecules or fragments thereof.

6. The method of claim 5, wherein at least some of said antibodies are Herceptin.

7. The method of claim 3, wherein said target cells are cancer cells.

8. The method of claim 1, further comprising the steps of:
preinjecting the individual with empty large liposomes; and
saturating the reticuloendothelial organs to reduce non-tumor specific spleen and liver uptake of said radionuclide upon adminstration thereof.

9. The method of claim 1, wherein said large liposomes have a diameter of about 600 nm to about 1000 nm.

10. The method of claim 1, wherein said large liposomes comprise molecules incorporated into outer membranes to stabilize said large liposomes.

11. The method of claim 10, wherein said stabilizing molecules are polyethyleneglycol-linked lipids (PEG-lipids).

12. The method of claim 10, wherein said stabilizing molecules are attached to an antibody, peptide, engineered molecule or fragment thereof.

13. The method of claim 1, wherein said large liposomes comprise a stabilizing agent incorporated therein or have an aqueous phase with a high pH thereby further facilitating retention of said radioactive decay intermediates.

14. The method of claim 13, wherein said stabilizing agent is a phosphate buffer, insoluble metal binding polymer, resin beads, metal-binding molecules or halogen binding molecules.

15. The method of claim 1, wherein said large liposomes comprise additional molecules, said molecules facilitating membrane fusion with target cells or facilitating endocytosis by target cells.

16. The method of claim 1, wherein said alpha particle emitting radionuclide is incorporated into the aqueous phase as a chelation compound.

17. The method of claim 1, wherein said alpha-particle-emitting radionuclide is ²²⁵Ac, ²²³Ra, ²¹³Bi, ²¹²Pb, or ²¹¹At.

18. A method of targeting cells in an individual for liposomal delivery of an alpha particle-emitting radionuclide thereto without systemic release of radioactive decay intermediates comprising the steps of:
entrapping said radionuclide within a small liposomal vesicle;
incorporating said radionuclide into the aqueous phase of large liposomes, said liposomes having a diameter sufficient to retain the radioactive decay intermediates of said radionuclide, said liposome comprising:
polyethyleneglycol-linked lipids (PEG-lipids) on outer membranes thereof; and
a targeting agent specific to the cells attached to the PEG-lipids; and
delivering said radionuclide to the cell whereby said targeting agents target the cells while said radioactive decay intermediates remain sequestered within said large liposomes.

19. The method of claim 18, further comprising the steps of:
preinjecting the individual with empty large liposomes; and
saturating the reticuloendothelial organs to reduce non-tumor specific spleen and liver uptake of said radionuclide upon delivery thereof.

20. The method of claim 18, wherein said large liposomes have a diameter of about 600 nm to about 1000 nm.

21. The method of claim 18, wherein said targeting agents are antibodies, peptides, engineered molecules or fragments thereof.

22. The method of claim 21, wherein at least some of said antibodies are Herceptin.

23. The method of claim 18, wherein said targeted cells are cancer cells.

24. The method of claim 18, wherein said large liposomes further comprise a stabilizing agent incorporated therein or have an aqueous phase with a high pH thereby further facilitating retention of said radioactive decay intermediates.

25. The method of claim 24, wherein said stabilizing agent is a phosphate buffer, insoluble metal binding polymer, resin beads, metal-binding molecules or halogen binding molecules.

26. The method of claim 18, wherein said large liposomes further comprise additional molecules, said molecules facilitating membrane fusion with target cells or facilitating endocytosis by target cells.

27. The method of claim 18, wherein said alpha particle emitting radionuclide is incorporated into the aqueous phase as a chelation compound.

28. The method of claim 19, wherein said alpha-particle-emitting radionuclide is selected from the group consisting of ²²⁵Ac, ²²³Ra, ²¹³Bi, ²¹²Pb, and ²¹¹At.
